(19) United States
(12) Patent Application Publication
(10) Pub. No.: US 2012/0071647 A1
(43) Pub. Date: Mar. 22, 2012

(54) Microfluidic reactor having an annular reaction chamber

(76) Inventors: Oliver Lade, Berlin (DE); Arno Steckenborn, Berlin (DE)

(21) Appl. No.: 13/322,819

(22) PCT Filed: May 12, 2010
(86) PCT No.: PCT/EP2010/056544

§ 371 Date: Nov. 28, 2011

(30) Foreign Application Priority Data
May 27, 2009 (DE) 10 2009 023 627.9

(51) Int. Cl. C07H 5/02; B01J 19/00

(52) U.S. Cl. 536/122; 422/159

(57) ABSTRACT

A microfluidic reactor has a reaction chamber which is configured as an annular channel. It is provided that segments have been formed in the annular channel such that a sequence of inlets and outlets (allows an alternation between the process fluids (A and B) to take place. Furthermore, particles which circulate in the annular channel and can be used, for example, for the adsorption and desorption of ions may be provided in the annular channel. In this way, the ions concerned can, for example, be extracted from the process fluid (A) and transferred into the process fluid (B). In a method, the reactor can be used for example for the purpose of obtaining ¹⁸F⁻ ions from water enriched therewith and feeding them to a solvent such as acetonitrile. In the further course of the method, the ¹⁸F⁻ ions can be used for producing a radiopharmaceutical (for example FDG).

### CROSS-REFERENCE TO RELATED APPLICATIONS

This application is based on and hereby claims priority to International Application No. PCT/EP2010/056544 filed on May 12, 2010 and German Application No. 10 2009 023 627.9 filed on May 27, 2009, the contents of which are hereby incorporated by reference.

### BACKGROUND

The invention relates to a microfluidic reactor having a reaction chamber which is configured as an annular channel that has openings for letting a process fluid in and out.

A microfluidic reactor of the type specified at the beginning is described, for example according to DE 102 24 150 B4. This reactor has an annular reaction chamber, in which an inlet and an outlet for a process fluid open out. In the annular chamber there may also be provided a multiplicity of particles, in particular magnetic particles, which can be moved in a circulating manner in the annular chamber by a magnetic drive. This accomplishes a mixing function, so that the reactions occurring in the annular chamber (of a chemical or physical nature) can be speeded up.

### SUMMARY

One potential object is to improve the microfluidic reactor having an annular channel to the extent that the reaction occurring in the annular chamber can be better supported.

Specifically, the inventors propose a microfluidic reactor with an annular channel being divided into a plurality of segments. The segments are formed by respectively having an inlet and an outlet for a process fluid, so that each segment can be charged separately with a process medium. Furthermore, respectively neighboring segments are in fluidic connection by way of the annular channel, so that a common reaction chamber is produced. Furthermore, the inlets and the outlets are provided with flow obstacles, though they are permeable to the process fluid. With the construction of the annular channel, it is advantageously ensured that a plurality of process fluids can be used simultaneously in the annular channel. On account of the dimensions of the annular channel as a microfluidic structure, i.e. dimensions which lie in the range around or below one μm, it is ensured that mixing together of the different process fluids can be at least largely avoided. Rather, the process fluids respectively flow from the entrance intended for them to the exit intended for them.

By using particles that are introduced into the annular chamber, a reactor designed in such a way can be used for enforcing chemical or physical reactions by way of the particles. In this case, the particles serve as carriers for constituents from the one process fluid, which can in this way be transferred to the other process fluid. For example, the particles may be coated with or be formed of an ion exchange resin, it being possible to take up ions from the one process fluid and give them up again in the other process fluid. This is a physical reaction. Alternatively, substances may also be bound by the particles and in this way transported from the one process fluid into the other process fluid, where they serve as chemical reactants for a reaction with substances from the other process fluid.

Since the particles are intended merely to serve as a vehicle for transporting constituents of the process fluids, but are not involved in the reaction themselves, they are confined in the annular channel, which is advantageously brought about by the particles being of a size that prevents them from being able to overcome the flow obstacles in the inlets and outlets. The transporting of the particles takes place, for example, by entrainment by the flowing process fluid.

According to a particular embodiment, it is provided that the particles are magnetic or magnetizable. This is advantageous whenever the reactor has a magnetic drive with which a magnetic field circulating in the annular channel can be generated. This is, for example, a permanent magnet, which is turned in the center of the annular channel by a motorized drive. Another possibility is that the annular channel is surrounded by electrical coils, which are switched off and on at certain time intervals. The precise way in which such magnetic drives operate is described in DE 102 24 150 B4. The magnetic particles are then entrained by the circulating magnetic field. A similar situation applies in the case of magnetizable particles, which are first magnetized by the magnetic field and then likewise entrained by the magnetic field. Magnetic particles which are coated with ion exchange resins are known per se and can, for example, be purchased from the company ORICA Watercare under the trade name MIEX®. Ferromagnetic iron particles may be used, for example, as magnetizable particles.

A further advantageous embodiment of the invention is obtained if a sluice for particles which has a closable opening with respect to the annular channel is provided. This opening must be sufficiently large to allow the particles that can be introduced into the sluice by way of another opening to be flushed out of the sluice into the annular channel by a fluid. In a similar way, the particles can also be flushed back again from the annular channel into the sluice by way of the opening, it thereby being advantageously possible to exchange the particles that are used and in this way prepare the proposed reactor for different reactions.

It is also advantageous if the inlets are fluidically connected to one another in groups in such a way that at least two process fluids can be alternately fed into the segments in a desired sequence. Thus, for example, every second inlet of the annular channel may be fed from one and the same feed line, while the other inlets are fed from another feed line. This makes it possible for the annular channel to be alternately charged with a first and a second process fluid. The interconnection may be established, for example, by suitable hose connections, which has the advantage that the interconnection can be changed without any problem by changing over the connection of the hoses. However, it is also particularly advantageous if the feed channels to the inlets are already provided in the desired interconnection in that component in which the annular channel is also provided. This is possible in the case of a layer-like construction of the microfluidic structure in a plurality of layers by appropriate introduction (for example etching) of depressions and by introduction of passages between the layers that connect these depressions.

For the reactor to operate continuously, the number of segments in the annular channel must be a multiple of the desired sequence of process fluids. In this case, not only is an alternate charging with two process fluids conceivable. It is also possible to use three or more process fluids in a specific sequence. Furthermore, it is also possible for a specific process fluid to be used more than once in a sequence. For example, it is conceivable that one segment is charged with the process fluid already mentioned above, which contains ions that are intended to be bound to the particles. There may then follow two segments for the second process fluid, to which the ions are intended to be given up again. This can achieve the effect that the desorption of the ions is achieved with a greater efficiency. In this case, a greater difference in concentration between the process fluid and the remaining ions to be desorbed on the particles is achieved in the second segment, seen in the direction of flow, into which likewise the second process fluid is fed. It goes without saying that it is also conceivable to provide the step of the adsorption of ions onto the particles in two successive segments if a sufficient absorption of ions would not otherwise take place.

In the same way as the inlets can meaningfully be fluidically connected to one another in groups, the outlets can also be interconnected accordingly. That which has been said with respect to the inlets applies correspondingly with regard to the combination of interconnections of the outlets. However, the outlets may also open out in groups, for example into a collecting tank, so that lines for carrying the process fluids away are not required.

It is particularly advantageous if the inlets are interconnected in such a way that a separating fluid can be fed in between segments which are intended to be charged with process fluids of different types that can mix with one another. For example, a conceivable sequence is one according to which firstly a first process fluid, then a separating fluid, then a second process fluid and then again a separating fluid are fed in, this sequence then being repeated. This advantageously avoids mixing together of the process fluids, though for the reasons already stated this is quite minor at the boundary surfaces. The separating fluid must be formed of a substance which cannot mix with either one or the other of the process fluids that are separate from one another. For example, the process fluids may be made hydrophilic and the separating fluid hydrophobic.

According to a particular embodiment of the annular reactor, the annular channel is configured with a constant cross section. This advantageously allows the process fluids to flow unhindered, allowing an exchange between the process fluids to be advantageously promoted by two openings being respectively located at the transitions between the segments, forming the inlet or outlet of the segment concerned and the inlet or outlet of the neighboring segment. The openings may lie directly opposite one another or be arranged slightly offset in relation to one another. Depending on the interconnection of the inlets or outlets, in this case one inlet and one outlet or two outlets or two inlets respectively lie opposite one another. In the event that one inlet and one outlet respectively lie opposite one another, the direction of flow is the same in the entire annular channel. In the case of this interconnection, it is not absolutely necessary for the particles to be conveyed by a magnetic drive. Rather, the particles are entrained by the flowing process fluids and respectively go over from one process fluid to the other, since they cannot leave the annular channel on account of the flow obstacles in the inlets and the outlets. However, it is also possible to make two inlets and two outlets respectively lie opposite one another. In this case, the direction of flow in the annular channel is reversed from segment to segment. The process fluids flow, as it were, toward one another or away from one another. In this case, in order to bring about an exchange of the particles from one process fluid into the other process fluid, a forced guidance of the particles must take place by the magnetic drive. It can be advantageously achieved thereby that the particles quickly run through the process fluid that is flowing in the same direction as the particles, while they take longer to run through the process fluid that is flowing against them. For example, a longer dwell time is obtained for the desorption of ions taking place in the counterflow and the counterflow principle applies to the giving up of the ions. This means that the particles that already have a low concentration of adsorbed ions are available to “fresh” process fluid, so that a concentration gradient is obtained, and a desorption still takes place on the particle surface in spite of the low concentration.

According to an alternative embodiment of the invention, it may be provided that the segments have a rhomboidal shape of the side surfaces with at the same time a constant channel height. In this case, the segments are respectively fluidically connected by way of opposing corners to the two neighboring segments, whereby the annular channel is produced. The two other corners respectively serve for receiving the inlet and the outlet, so that the direction of flow of the process fluid in the segments takes place transversely in relation to the direction of movement of the particles. In the region of the annular channel, the rhomboidal segments have the greatest cross section with respect to the direction of through-flow by the process fluid, so that here the flow velocity is very slow and there is time for the flowing particles to be able to support the desired physical or chemical reaction.

The inventors also propose a method for operating a reactor which has a comparatively great efficiency with regard to the desired reaction.

Specifically, the inventors propose using in the reactor particles of which the surface is used as an ion exchanger and which circulate in the annular channel, a process fluid from which ions are bound to the particles and then a process fluid to which the ions that are bound to the particles are given up again being alternately fed in. By this method, the advantages already explained in conjunction with the reactor are achieved.

According to one embodiment of the method, it is provided that a separating fluid is respectively fed in between the two process fluids. As already explained, this allows mixing of the two process fluids to be prevented.

It is also particularly advantageous if the one process fluid includes water in which ¹⁸F⁻ ions that become attached to the particles are dissolved. It is also advantageous if another process fluid that is used is one which includes an acetonitrile (hereafter MeCN, Me standing for methyl) to which ¹⁸F⁻ ions that are attached to the particles are given up. This is a reaction that is necessary to extract ¹⁸F for the production of radioactive contrasting agents and make them available to further reactions. The reactor operates here as an ion exchanger, that is to say as a device for carrying out an ion exchange, whereby a reconcentration of ¹⁸F is used in the production of, for example, ¹⁸F⁻FDG (more about this hereafter). In this case, the ion exchange with the particles takes place from a solution of ¹⁸F⁻ into H₂¹⁸O, which originates from a cyclotron. The desorption of ¹⁸F from the particles and the regeneration thereof may take place in distilled laboratory water or in acetonitrile (MeCN). In this case, there is a fundamental distinction between three basic steps: the charging of particles with ¹⁸F⁻ ions by the ion exchange resin located on them, the separation of this ion exchange resin from the solution (H₂¹⁸O) and the regeneration of the particles. As a result of the continuous operating mode of the reactor, these temporally successive process steps are thus carried out simultaneously, but spatially separate from one another. Therefore, the ion exchange resin must also be accommodated on the particles in a spatially variable manner. The separation is ensured by the segments of the annular channel. The processes in a partial region of the reactor that occur simultaneously in the further sequence of the segments in the reactor can be described as follows. This description should be understood as given merely by way of example and without restricting generality.

There are three types of segments. Into the first segment there flows H₂¹⁸O solution with ¹⁸F⁻ ions dissolved therein, originating from the cyclotron. Here an adsorption of the ions takes place on the ion exchange particles, which in this way are charged with ¹⁸F⁻. The particles leave the segment and pass into a second segment with a separating fluid, where no reactions take place. The H₂¹⁸O freed of the ¹⁸F⁻ ions flows away through the exit of the first segment. The ion exchange particles subsequently pass into the third segment, where MeCN is fed in. The MeCN takes up the ¹⁸F⁻ ions, which are desorbed from the ion exchange particles. The ion exchange particles largely freed of the ¹⁸F⁻ ions then pass again into a fourth segment with a separating fluid. The separating fluid is formed of a liquid which cannot be mixed with either water or MeCN.

Mineral oils or natural oils come into consideration for example here. The entire process described is repeated a plurality of times in the annular channel, where the number of segments that are present in the annular channel should advantageously be a multiple of these four basic steps.

An alternative solution for achieving the object directed at the method is obtained if particles of which the surface is used as a catalyst and which circulate in the annular channel are used in the reactor, a process fluid in which the catalysis of a reaction is carried out and a process fluid in which a regeneration of the particles is carried out being alternately fed in. This alternative of the solution can be used if the particles used do not allow a sustained catalysis of the desired reaction. To maintain the catalytic reaction for a sustained time, it is then necessary that the catalyst, that is to say the surface made available by the particles, regularly undergoes a regeneration. Selected for this purpose is a suitable process fluid, which the particles run through once their activity in the process fluid in which the catalysis of the reaction is intended to take place has lessened or even ceased.

A further application for particles that are used as a catalyst is that particles of which the surface is used as a phase transfer catalyst are used. Phase transfer catalysts serve for supporting phase transfer catalysis (PTC). This is understood as meaning a chemical process in which the reactants of a desired reaction are present in at least two non-miscible phases (process media), the phase transfer catalyst enabling one of the reactants to pass through the phase boundary into that phase, in which the chemical process proceeds. The phases are, for example, water and an organic solvent. The transferred reactant may, for example, be an ion that does not dissolve in the organic phase and has to be transported from the aqueous phase into the organic phase. Magnetic particles may also be used here, their use for this purpose being known in principle

### BRIEF DESCRIPTION OF THE DRAWINGS

These and/or other aspects and advantages will become apparent and more readily appreciated from the following description of the embodiments, taken in conjunction with the accompanying drawings of which:

FIG. 1 shows by way of example a construction with which FDG marked with ¹⁸F can be obtained, an exemplary embodiment of the proposed reactor being used for the separation of ¹⁸F⁻ ions from H₂¹⁸O and admixture with MeCN,

FIGS. 2 and 3 show as a schematic view exemplary embodiments of the proposed reactor, which can be integrated into the construction according to FIG. 1 in a way corresponding to detail X.

### DETAILED DESCRIPTION

Reference will now be made in detail to the embodiments, examples of which are illustrated in the accompanying drawings, wherein like reference numerals refer to the like elements throughout. The embodiments are described below to explain the present invention by referring to the figures.

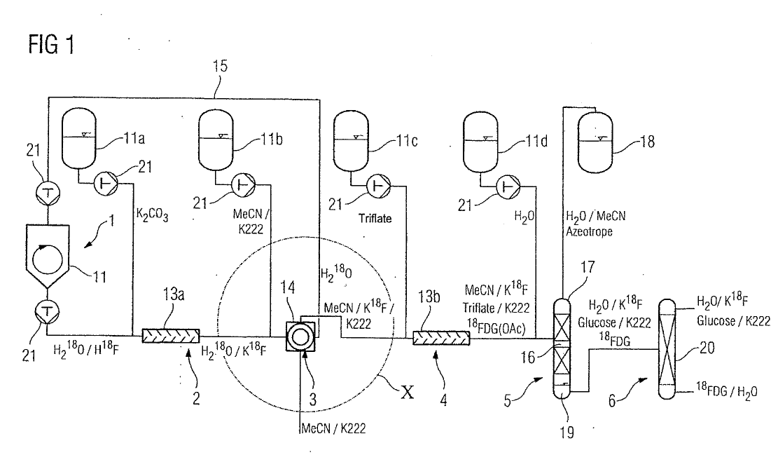

In FIG. 1 there is an installation for carrying out a method for producing a radiopharmaceutical ¹⁸FDG (this is the abbreviation for 2-deoxy-2-¹⁸fluoro-D-glucose). The six parts of the process necessary for this, steps 1 to 6, can be taken from FIG. 1, since they are respectively carried out simultaneously in a certain part of the continuously flowed-through apparatus (i.e. a reference volume under consideration in the continuous flow passes through the six parts of the process, one after the other). The fluid flow is maintained by pumps 21.

In part 1, the irradiation of the target H₂¹⁸O takes place in a cyclotron 11. In this cyclotron, the target is enclosed in a reaction vessel, which may also be configured as a through-flow cell for the purpose of continuous charging and removal of the target. In the cyclotron, the production of the radionuclide ¹⁸F, which is present in a dissolved state in the H₂¹⁸O, takes place. In this case, the ¹⁸O of the water is converted into ¹⁸F by irradiation with an alpha particle. In this way, hydrofluoric acid (H¹⁸F) dissolved in the H₂¹⁸O is obtained.

In part 2, potassium carbonate K₂CO₃ is admixed with this solution from a storage tank 11a, complete mixing together taking place by a micromixer 13a. This produces dissolved potassium fluoride K¹⁸F, which in part 3 can be separated from the H₂¹⁸O. This takes place in the way already described by the proposed reactor 14, which is used to bound by way of ion exchange particles the ¹⁸F⁻ from the H₂¹⁸O, forming the first process fluid, and give them up to acetonitrile MeCN. The H₂¹⁸O can be returned to the cyclotron 11 by way of the line 15. In this case, the (low) consumption of H₂¹⁸O can be compensated by the nuclear reaction if the process is intended to proceed continuously for a sustained time.

The acetonitrile MeCN is fed to the reactor as the second process fluid, this representing an aprotic, polar solvent. At the same time Kryptofix® (hereafter K222 for short) is fed in as a solubilizer, this substance dissolving ¹⁸F⁻ in the form of a complex in the MeCN.

Furthermore, in a way not represented any more specifically, a separating fluid may be respectively fed into the reactor 14 between the first process fluid and the second process fluid. The detailed way in which the reactor 14 operates is described more specifically in the further figures.

In part 4, there is a reaction of the K¹⁸F with triflate, which provides a precursor for the product ¹⁸FDG. The triflate is added from a storage tank 11c. Triflate is a mannose derivative which is suitable for the desired reaction of the type constituting a nucleophilic substitution with ¹⁸F⁻. This reaction is supported by rapid mixing together of the added triflate with the reaction solution, this being brought about in a micromixer 13b. A further functionality in this part of the process is the temperature control of the reaction mixture. This takes place, for example, by the micromixer containing not only channels which are flowed through by the reaction mixture but also channels which are flowed through by a temperature control fluid (not represented). This causes the heat exchange between the reaction mixture and the temperature control fluid.

In part 5 there is a further solvent change from MeCN to water (H₂O). For this purpose, H₂O is fed in from a storage tank 11d. The solvent change is carried out by an azeotropic reactive rectification. Used for this purpose is a rectification column 16, at the head 17 of which the azeotropic mixture, formed of H₂O and MeCN, can be removed and fed to a waste tank 18. For this purpose, the aprotic, polar solvent must be more readily volatile than water or form a low-boiling azeotrope in the present mixture with water.

A hydrolysis reaction is simultaneously carried out in a rectification column for splitting off protective groups that protect the otherwise unprotected hydroxy groups of the triflate from an unwanted reaction with ¹⁸F⁻. This reaction is acidically or alkalinically catalyzed (for example an acidic packing with the catalyst Amberlyst 15 may be used). After carrying out the hydrolysis, H₂O, remains of K¹⁸F and glucose as a byproduct of the reaction, K222 and the desired reaction product, the radiopharmaceutical ¹⁸FDG, can be found in the sump.

The radiopharmaceutical must be separated from the other constituents in a further reaction step by a simulated moving bed apparatus (SMB apparatus). In the SMB apparatus, a chromatographic process can be continuously carried out, so that the desired reaction product can be continuously removed in water. Subsequent purifying processes (not represented) can be carried out to improve the product quality.

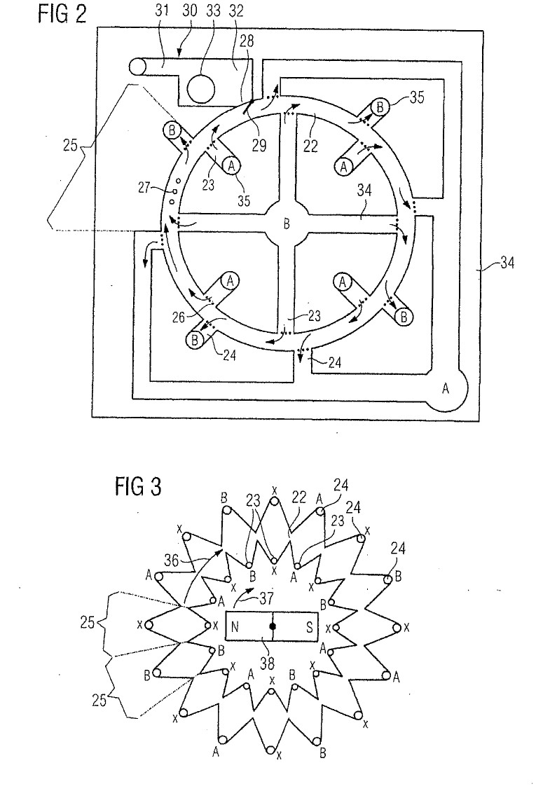

In FIG. 2, a plan view of a microfluidic reactor can be seen. This includes various stacked layers, only that layer in which an annular channel 22 is formed as a reaction chamber being represented in FIG. 2 (as a plan view). In this annular channel, inlets 23 and outlets 24 also open out, these inlets and outlets respectively being arranged opposite one another with a small offset. Between the inlets 23 and outlets 24 respectively lying substantially opposite one another there are the boundaries between the individual segments 25, which are respectively charged with a process fluid A and a process fluid B. Which inlets and outlets are respectively intended for the process fluid A and the process fluid B can be seen from the letters depicted in FIG. 1, the respective direction of flow of the process fluid to the inlets and outlets 23, 24 being indicated.

At the inlets and outlets 23, 24 there are also flow obstacles 26, which though permeable to the process fluids A, B are not permeable to particles 27, which are captured in the annular channel 22. These can only be introduced into the annular channel 22 or removed from it by way of an opening 28, which can be closed by a valve 29.

To accomplish the introduction and removal of the particles, a sluice 30 is provided downstream of the valve 29. This sluice has an inlet or outlet 31, flushing through of the sluice chamber 32 of the sluice being possible thereby. Before the flushing through, the particles can be introduced into the sluice chamber by way of a further opening 33, in order subsequently to be flushed into the annular channel 22.

The layer of the microfluidic reactor that is represented in FIG. 2 is simultaneously used for interconnecting the entrances for the process fluid B and the exits for the process fluid A by a tree-like structure of channels 34. On the other hand, the entrances for the process fluid A and the exits for the process fluid B are connected by way of passages 35 to a layer that is not represented, which lies below the layer that is represented and has a comparable tree-like system of channels for interconnecting the corresponding entrances and exits.

According to FIG. 3, a different configuration of the annular channel 22 is represented. The segments 25 are of a rhomboidal configuration, the annular channel being produced by the rhomboids being respectively connected at the opposing corners to the neighboring rhomboids. The path of the particles through the annular channel is indicated by an arrow 36. The rotational sense in the movement of the particles corresponds to the rotational sense 37 of a magnetic drive, which includes a rotatable drivable magnet 38.

The inlets 23 and outlets 24 of the segments 25 respectively lie in the other two corners of the rhomboids and, as described in relation to FIG. 2, are given the form of passages. It will be clear that the sequence of the charging of the segments proceeds in accordance with the following scheme. A process fluid A, a separating fluid X, a process fluid B and a separating fluid X are respectively fed in alternately. This sequence is repeated four times around the circumference of the annular channel. The direction of flow of the process fluids A, B and the separating fluid X is directed radially outward. However, an interconnection in which the direction of flow is reversed is equally conceivable. It is also possible, for example, for the process fluid A, B to flow radially outward, while the separating fluid X flows radially inward.

The interconnections of the segments according to FIG. 2 and according to FIG. 3 are given merely by way of example. Other interconnections are also conceivable, with some of the possibilities now being given. The process fluids are merely designated here by capital letters and the separating fluid by X. Without restricting generality, the following sequences for the charging of the segments are conceivable.

### A-A-B

### A-A-X-B-X

### A-X-B-B-X

### A-X-B-X-C-X

### A-B-X-C-X

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1-15. (canceled)

16. A microfluidic reactor comprising:
a reaction chamber configured as an annular channel divided into a plurality of segments directly adjacent one another, each segment having a first end and a second end, wherein
each segment has an inlet at the first end to allow fluid to enter the segment,
each segment has an outlet provided at the second end to allow fluid to exit the segment,
neighboring segments are connected by way of the annular channel, and
the inlets and the outlets are provided with respective flow obstacles that are permeable to a respective desired process fluid.

17. The reactor as claimed in claim 16, wherein particles which cannot pass the flow obstacles are confined in the annular channel.

18. The reactor as claimed in claim 17, wherein the particles are magnetic or magnetizable.

19. The reactor as claimed in claim 18, wherein
the reactor has a magnetic drive that generates a magnetic field,
the magnetic field circulates around the annular channel, and
the magnetic field circulates fluid through the annular channel by circulating the particles.

20. The reactor as claimed in claim 16, wherein
the reactor has a magnetic drive that generates a magnetic field, and
the magnetic field circulates around the annular channel.

21. The reactor as claimed in claim 17, further comprising:
a sluice to move the particles into and out of the annular channel, the sluice having a closable opening that connects the sluice to the annular channel.

22. The reactor as claimed in claim 16, wherein
the annular channel has at least four inlets, with at least two groups of two inlets, and
within each group, the inlets are fluidically connected to one another in such a way that at least first and second process fluids can be alternately fed into the segments in a desired sequence.

23. The reactor as claimed in claim 22, wherein
the annular channel has at least eight inlets, with at least four groups of two inlets,
the first and second process fluids are different from one another, but mixable, and
within each group, the inlets are fluidically connected to one another in such a way that a separating fluid can be fed in between segments charged with the first and second process fluids.

24. The reactor as claimed in claim 16, wherein the annular channel has a constant cross section.

25. The reactor as claimed in claim 24, wherein
a transition area is provided between each pair of first and second neighboring segments, and
each transition area has the inlet of the first neighboring segment and the outlet of the second neighboring segment.

26. The reactor as claimed in claim 16, wherein
the segments each have side surfaces with a rhomboidal shape,
the rhomboidal shape has first opposing corners and second opposing corners,
at the first opposing corners, each segment is fluidically connected to neighboring segments, and
at the second opposing corners, the inlet and the outlet are respectively provided.

27. A method for operating a microfluidic reactor, comprising:
providing particles having an ion exchange surface;
circulating the particles through an annular channel divided into a plurality of segments directly adjacent one another and connected by way of the annular channel, each segment having a first end and a second end, with an inlet provided at the first end to allow fluid to enter the segment and an outlet provided at the second end to allow fluid to exit the segment;
feeding a first process fluid to at least one first inlet;
using the particles to receive ions from the first process fluid;
feeding a second process fluid to at least one second inlet; and
using the second process fluid to receive ions from the particles.

28. The method as claimed in claim 27, further comprising feeding a separating fluid between the first and second process fluids.

29. The method as claimed in claim 27, wherein
the first process fluid is water having F⁻ ions dissolved therein, and
the ¹⁸F⁻ ions become attached to the particles.

30. The method as claimed in claim 27, wherein the second process fluid is MeCN that receives ¹⁸F⁻ ions from the particles.

31. A method for operating a microfluidic reactor, comprising:
providing particles having a catalyst surface;
circulating the particles through an annular channel divided into a plurality of segments directly adjacent one another and connected by way of the annular channel, each segment having a first end and a second end, with an inlet provided at the first end to allow fluid to enter the segment and an outlet provided at the second end to allow fluid to exit the segment;
feeding a first process fluid to at least one first inlet;
using the particles to catalyze a reaction with the first process fluid;
feeding a second process fluid to at least one second inlet; and
using the second process fluid to regenerate the catalyst.

32. The method as claimed in claim 31, further comprising feeding a separating fluid between the first and second process fluids.

33. The method as claimed in claim 31, wherein the particles have a phase transfer catalyst surface.
